(19) United States
(12) Patent Application Publication
(10) Pub. No.: US 2021/0236167 A1
(43) Pub. Date: Aug. 5, 2021

(54) DELIVERY DEVICE FOR BIOLOGIC MATERIAL

(71) Applicant: Artoss, Inc., Saint Cloud, MN (US)

(72) Inventors: Paul J. BYERLY, Temecula, CA (US); Nirav AMIN, Yorba Linda, CA (US); Philip ALEI, Carlsbad, CA (US)

(73) Assignee: Artoss, Inc., Saint Cloud, MN (US)

(21) Appl. No.: 17/164,517

(22) Filed: Feb. 1, 2021

Related U.S. Application Data
(60) Provisional application No. 62/968,872, filed on Jan. 31, 2020.

(51) Int. Cl. A61B 17/34

(52) U.S. Cl. CPC A61B 17/3472; A61B 2017/0042; A61B 17/3421

(57) ABSTRACT

The present disclosure relates to a novel and advantageous delivery device for delivering biologic material. Particularly, the present disclosure relates to a novel and advantageous cannula and hub assembly for delivery of biologic material to a surgical site. More particularly, the present disclosure relates to a novel and advantageous cannula and hub assembly for delivery of biologic material for surgical treatment of subchondral bone defects.

### CROSS REFERENCE TO RELATED APPLICATION

The present application claims the benefit of U.S. Provisional patent Application No. 62/968,872, filed on 31 Jan. 2020, the entire contents of which are incorporated herein by references.

### FIELD OF THE INVENTION

The present disclosure relates to a novel and advantageous delivery device for delivering biologic material. Particularly, the present disclosure relates to a novel and advantageous cannula and hub assembly for delivery of biologic material to a surgical site. More particularly, the present disclosure relates to a novel and advantageous cannula and hub assembly for delivery of biologic material for surgical treatment of subchondral bone defects.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The surgical repair of subchondral defects, sometimes referred to as subchondroplasty, is a minimally-invasive surgery that targets and treats subchondral defects associated with chronic bone marrow lesions in patients with knee osteoarthritis or insufficiency fractures. Bone marrow lesions, also referred to as subchondral bone marrow edema, are an often-painful defect of the spongy cancellous bone that underlies and supports the cartilage of a joint. In the setting of knee osteoarthritis, patients with subchondral bone marrow edema often undergo faster joint degeneration and experience more pain than those without bone marrow edema. The subchondroplasty procedure may be performed along with arthroscopy for visualization and treatment of findings inside the joint. In some cases, an open or mini-open procedure is necessary for access to the defect.

In patients with knee osteoarthritis, subchondroplasty is most frequently performed to treat bone marrow edema in the medial compartment (i.e., medial tibia plateau, medial femoral condyle, or both for “kissing” contrecoup lesions). Other possible target sites for subchondroplasty include the lateral compartment, patella, and trochlea.

During subchondroplasty, the surgeon injects synthetic bone substitute, such as calcium phosphate, into the region of bone marrow edema to fill the intertrabecular space, thereby providing local mechanical support to the bone at that site. This injection preserves preexisting trabeculae and may induce improved bone remodeling.

Cannula systems have been used to deliver synthetic bone substitute/calcium phosphate or other materials to a variety of bones including, but not limited to: vertebral bodies, distal and proximal tibia, distal and proximal femur. Zimmer, Inc. and Arthrex, Inc. currently have such devices for use in delivering bone substitute material to long bones.

Using currently available systems, it is necessary for a surgeon to visually align the delivery device to ensure proper orientation of a cannula for delivery of biologic material. Further, many of these systems are high profile and portions of the device may catch on materials in the operating room. Further, because of the high profile nature, a further device must be used to pinch tissue away from the device.

Thus, there is a need in the art for low profile subchondroplasty system that provides a tactile indication of positioning of the device within the patient.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a delivery device for delivering biologic material. The delivery device may comprise a push rod assembly, a cannula assembly, and a connection and alignment piece for detachably connecting the push rod assembly and the cannula assembly. In one embodiment, the cannula assembly comprises a cannula, wherein biologic material may be directed through the cannula and out of an opening provided at or near a distal end of the cannula. The connection and alignment piece may include an alignment mechanism and a connector. The alignment mechanism may be configured to tactilely index a location of the opening. The connector may be configured for engagement with a syringe.

In another embodiment, a further delivery device for delivering a biologic material is provided. The delivery device may include a cannula assembly and a push rod assembly. The push rod assembly may include a push rod and a hub. The cannula assembly may include a cannula, a connection and alignment piece, and a latching mechanism. The cannula may be closed and have a trocar tip. Biologic material may be directed through the cannula and out of an opening provided on a surface of the cannula at or near a distal end of the cannula. The connection and alignment piece may have an alignment ridge and a connector. The alignment ridge may be configured to tactilely index a location of the opening. The connector may be configured for engagement with a syringe. The connection and alignment piece may include a latching mechanism for releasably engaging the push rod assembly.

In yet another embodiment, a method for delivering a flowable material comprising providing a delivery device and using the delivery device to deliver the material. The delivery device may include a push rod assembly, a cannula assembly, and a connection and alignment piece for detachably connecting the push rod assembly and the cannula assembly. In one embodiment, the cannula assembly comprises a cannula, wherein biologic material may be directed through the cannula and out of an opening provided at or near a distal end of the cannula. The connection and alignment piece may include an alignment mechanism and a connector. The alignment mechanism may be configured to tactilely index a location of the opening. The connector may be configured for engagement with a syringe. Using the delivery device may comprise joining the push rod assembly and the cannula assembly, driving the cannula into bone, releasing the push rod assembly from the cannula assembly, engaging the syringe with the connector of the cannula assembly, using the alignment piece to align the opening with an area of bone to be treated, and expelling material from the syringe to the area of bone.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a illustrates an outside view of a partially assembled delivery device, in accordance with one embodiment.

FIG. 1b illustrates a cross sectional view of the delivery device of FIG. 1a.

FIG. 2a illustrates an outside view of an assembled delivery device, in accordance with one embodiment.

FIG. 2b illustrates a cross sectional view of the delivery device of FIG. 2a.

FIG. 2c illustrates an alternate view of the delivery device of FIG. 2a.

FIG. 2d illustrates an alternate view of the delivery device of FIG. 2a.

FIG. 2e illustrates an alternate view of the delivery device of FIG. 2a.

FIG. 3a illustrate a cross sectional view of a push rod assembly for a delivery device, in accordance with one embodiment.

FIG. 3b illustrate a cross sectional view of a portion of a push rod assembly for a delivery device, in accordance with one embodiment.

FIG. 4 illustrates an external view of a hub of a push rod assembly for a delivery device, in accordance with one embodiment.

FIG. 5a illustrates a push rod and fixation element of a push rod assembly of a delivery device, in accordance with one embodiment.

FIG. 5b illustrates a push rod and fixation element of a push rod assembly of a delivery device, in accordance with one embodiment.

FIG. 6a illustrates an outside view of a cannula assembly of a delivery device, in accordance with one embodiment.

FIG. 6b illustrates a cross sectional view of the cannula assembly of FIG. 6a.

FIG. 7 illustrates a proximal end of a cannula of a cannula assembly of a delivery device, in accordance with one embodiment.

FIG. 8 illustrates a connection and alignment piece fit to a proximal end of a cannula of a cannula assembly of a delivery device, in accordance with one embodiment.

FIG. 9 illustrates a hub of a push rod assembly of a delivery device and a connection and alignment piece of a cannula assembly of delivery device, before attachment, in accordance with one embodiment.

FIG. 10 illustrates a view of a hub of a push rod assembly and a connection and alignment piece of a cannula assembly of a delivery device, after attachment, in accordance with one embodiment.

FIG. 11 illustrates an alternative embodiment of a hub and toggle of a delivery device, in accordance with one embodiment.

FIG. 12 illustrates an end view of a hub assembly, in accordance with one embodiment.

FIG. 13 illustrates an assembled delivery device, in accordance with one embodiment.

### DETAILED DESCRIPTION

The present disclosure relates to a novel and advantageous delivery device for biologic material. Particularly, the present disclosure relates to a novel and advantageous cannula and hub assembly for delivery of biologic material, such as bone cement, for surgical treatment of subchondral bone defects. It is to be appreciated that all dimensions and materials suggested herein are exemplary only and not intended to be limiting.

Delivery Device for Delivering Biologic Material

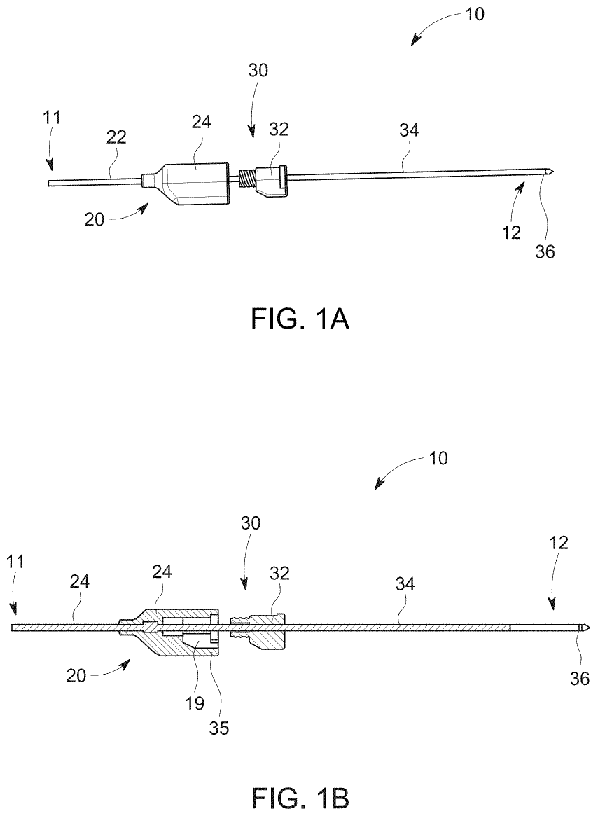

FIGS. 1a and 1b illustrate a partially assembled delivery device in accordance with one embodiment. The delivery device may be used to delivery biologic material, such as synthetic bone substitute/calcium phosphate or other material. FIG. 1a illustrates an outside view of the delivery device 10. FIG. 1b illustrates a cross sectional view of the delivery device 10. In the embodiment shown, the delivery device comprises a push rod assembly 20 and a cannula assembly 30. FIGS. 1a and 1b illustrate the delivery device before the push rod assembly 20 and cannula assembly 30 are joined by receipt of a connection and alignment piece 32 of the cannula assembly 30 by a receptacle 19 of the push rod assembly 20. Any suitable material may be used to form the various components of the delivery device. In some embodiments a polymer material is used. In other embodiments a metal material may be used. In some embodiments, some components may be formed substantially of a polymer material and other components may be formed substantially of metal. Forming the various components may be done by molding, injection molding, or other.

In the embodiment shown, the push rod assembly 20 comprises a push rod 22 and a hub 24, the hub having a receptacle 19. The cannula assembly comprises a connection and alignment piece 32 and a cannula 34. The connection and alignment piece 32 may have complementary cross sections such that at least a portion of the connection and alignment piece 32 is received by the receptacle 19. These may connect via a male-female connection or via any other suitable shape or connection. The shape of the hub 24 may be selected such that it is generally easy to determine the orientation of the device using only touch In one embodiment, the connection and alignment piece 32, the receptacle 19, and the hub 24 may have a tear dropped cross section.

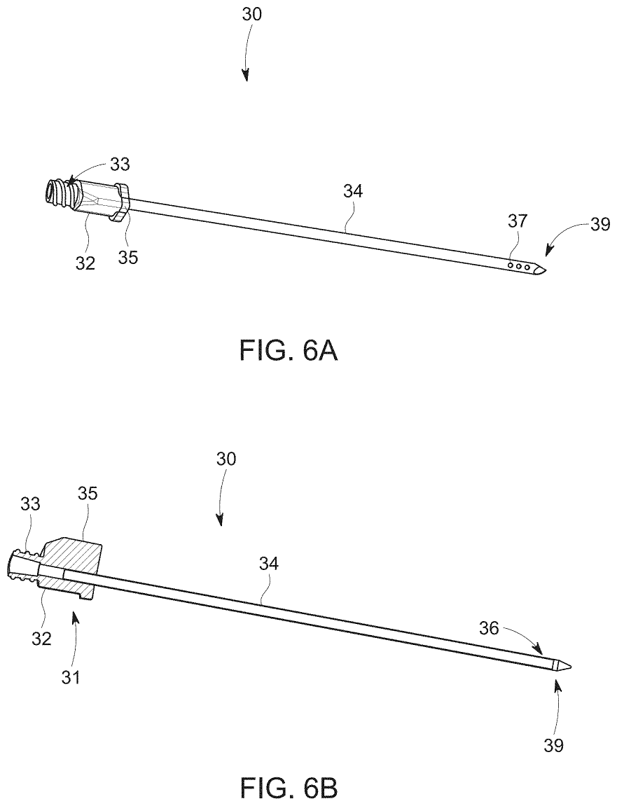

The connection and alignment piece 32 may include an alignment mechanism configured to tactilely index a location of an opening in the cannula. For example, the connection and alignment piece may include an alignment ridge 35 that indexes openings 37 (shown in FIG. 6a) of the cannula 34. The openings are exit holes for delivery of material. The material may be a viscous biologic material such as synthetic bone cement. Any suitable number of holes may be provided and such holes may be provided in any suitable orientation. The alignment ridge gives the physician a tactile and visual indication of alignment of the openings 37.

The delivery device 10 has a proximal end 11 and a distal end 12. During use, the proximal end 11 is generally towards the surgeon and the distal end 12 is towards the patient. A wire piece 22 may be providing extending proximally from the hub 24. The distal end 12 of the delivery device 10 may be the distal end of the cannula assembly 30. The distal end 12 may be open or may be closed and have openings, described more fully below. Biologic material for delivery by the delivery device 10 may flow through the delivery device and out at or proximate the distal end, either by way of an open end or by way of openings in the distal end. A radiopaque marker 36 may be provided at or near the distal end 12. In one embodiment, the radiopaque marker 36 is a tantalum bar. The radiopaque marker 36 may facilitate proper indexing and visualization under x-ray examination/fluoroscopy.

In one embodiment, the total length of the delivery device is approximately 8.3 inches with the proximal extension of the push rod from the hub being approximately 4.8 inches. In other embodiments, the total length of the delivery device may range from approximately 6 inches to approximately 11 inches, with the proximal extension of the push rod from the hub ranging from approximately 2 inches to approximately 7 inches. In alternative embodiments, any suitable length may be used.

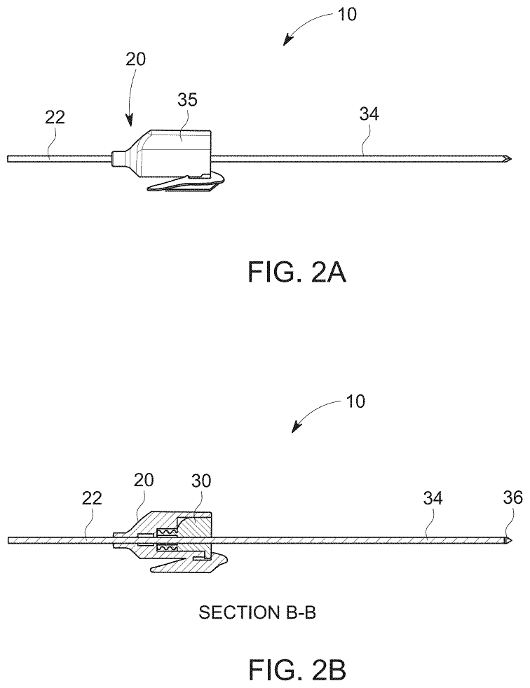

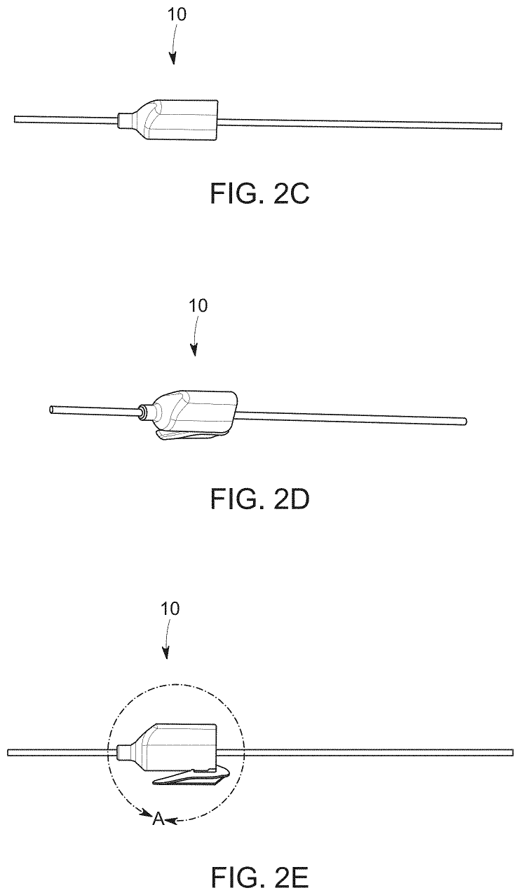

FIGS. 2a and 2b illustrate the delivery device 10 of FIGS. 1a and 1b as assembled with the push rod assembly 20 and cannula assembly 30 joined. FIG. 2a illustrates an outside view of the delivery device 10. FIG. 2b illustrates a cross sectional view of the delivery device 10. FIGS. 2c-2e illustrate alternative views of the assembled delivery device 10. As assembled, a cannula 34 of the cannula assembly 30 extends distally and a push rod 22 of the push rod assembly 20 extends proximally. In the embodiment shown, a hub 24 of the push rod assembly 20 has a receptacle 19 for receiving a connection and alignment piece of 32 of the cannula assembly 30. The receptacle 19 and the connection and alignment piece 32 may thus have complementary shapes such as a male-female connection.

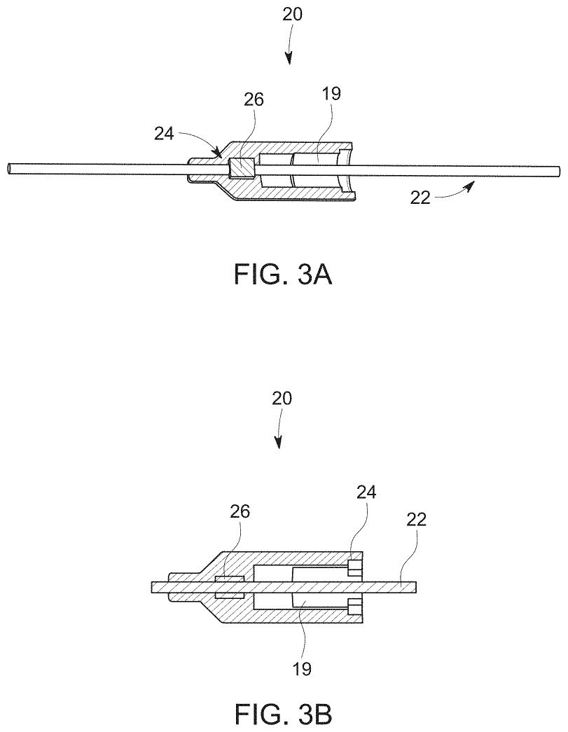

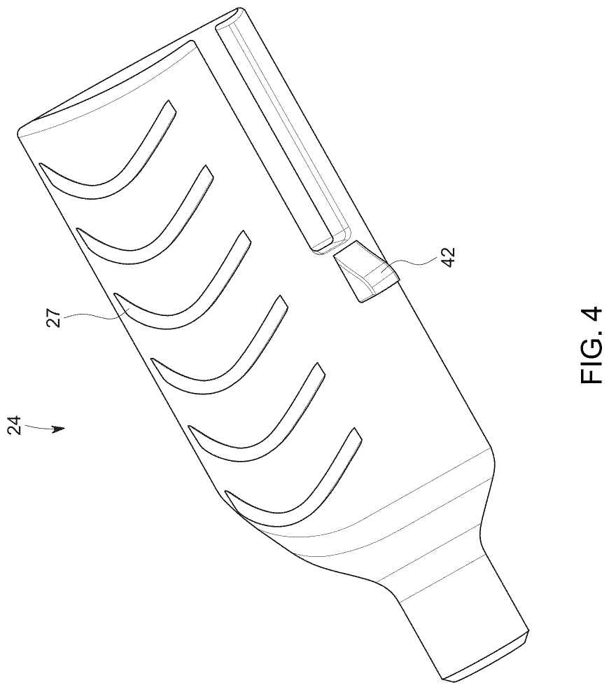

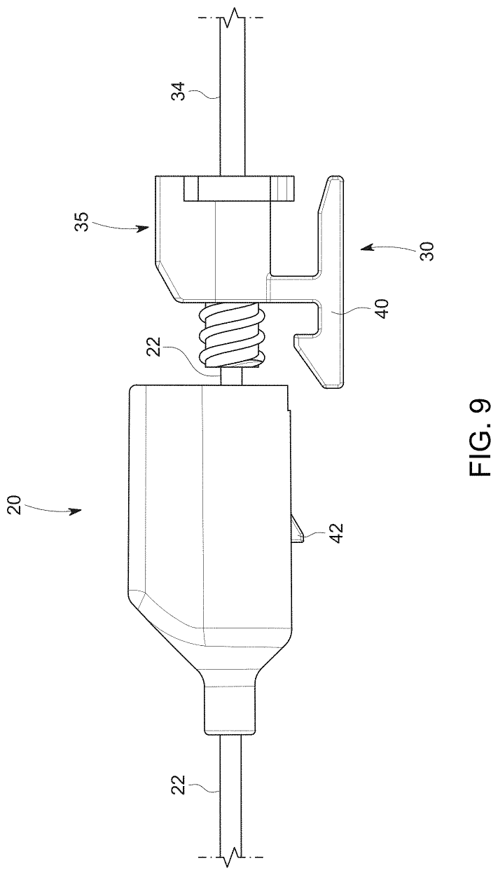

FIGS. 3a and 3b illustrate cross sectional views of a push rod assembly 20 for a delivery device, in accordance with one embodiment. In the embodiment shown, the push rod assembly 20 comprises a push rod 22 and a hub 24. The push rod 22 may comprise a heavy gauge wire, such as a k-wire, and a fixation element 26. The push rod 22 may have a diameter of between about, for example, 0.05 and 0.15 inches. In one embodiment the push rod 22 may have a diameter of about 0.090 inch. The push rod may extend proximally and distally from the hub 24. The hub may be formed from a plastic material. FIG. 4 illustrates an external view of the hub 24. As shown, the hub 24 may include grip features 27 and a catch 40, described further with respect to FIG. 9.

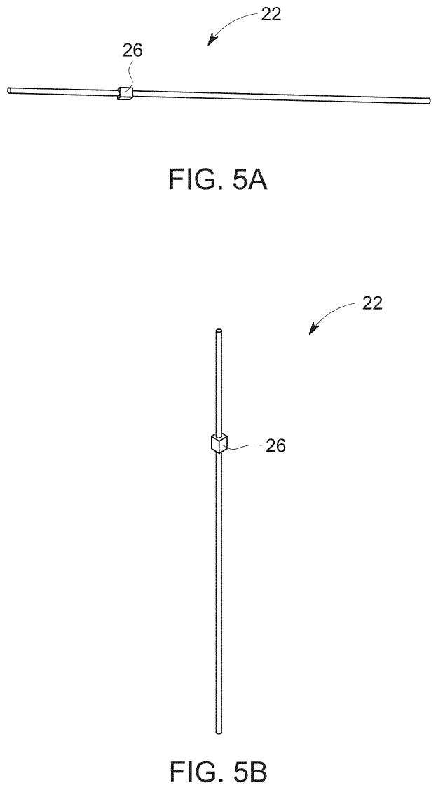

FIGS. 5a and 5b illustrate a push rod 22 and fixation element 26 of a push rod assembly for a delivery device, in accordance with one embodiment. The fixation element 26 operates to fix the hub on the push rod 22. The fixation element 26 may be braised onto the push rod 22 and molded into the hub 24. The push rod 22 and the fixation element 26 may be formed of the same material or may be formed of different materials. In the embodiment shown, the fixation element 26 is a cube. The cube 26 may have dimensions of, for example, 0.25 inch long, and 0.188 inch wide. In alternative embodiments, other dimensions may be used, for example between about 0.20 and 0.30 inch long and between about 0.14 and 0.20 inch wide. In some embodiments, a fixation element 26 may not be provided and other means for fixing the hub onto the push rod 22 may be used.

FIGS. 6a and 6b illustrate a cannula assembly 30 of a delivery device, in accordance with one embodiment. FIG. 6a illustrates an outside view of the cannula assembly 30. FIG. 6b illustrates a cross sectional view of the cannula assembly 30. As shown, the cannula assembly may include a connection and alignment piece 32 and a cannula 34. As is described more fully below, the alignment piece can be used to facilitate a surgeon orienting the delivery device in a desired orientation for expelling material from the device. The connection and alignment piece 32 includes a connector 33 and an alignment ridge 35. The connector may be configured for engagement with a syringe. In one embodiment, the connector 33 comprises male threading, or luer lock threading, configured to engage female threading of a luer locking syringe. Other manners of fixing the connector to a syringe may alternatively be used. The cannula 34 includes a proximal end 31 engaging with the connection and alignment piece 32 and a distal end 39 for insertion into a patient. A radiopaque marker 36 may be provided at or near the distal end 39.

Biologic material, such as synthetic calcium phosphate, is directed through the cannula 34 and generally out of or proximate to the distal end 39. The distal end 39 thus may be configured to allow the biologic material to escape from the delivery device. This may be done by providing an open distal end or by providing openings in the distal end. The embodiment of FIG. 6a illustrates openings 37 in the cannula 34. In embodiments where openings are provided at or near the distal end 39 of the cannula 34, the cannula 34 may be referred to as a fenestrated cannula. As shown, the distal end 39 may be pointed to pierce bone. For example, the distal end 39 may have a trocar tip having a sharp pyramidal shape to pierce bone.

In an embodiment wherein the distal end 39 of the cannula 34 is open, the viscous material flows out of the open distal end. The push rod of the push rod assembly may be provided with a trocar tip to pierce bone.

FIG. 6a shows alignment of the connection and alignment piece 32 with the openings 37 in the distal end 39 of the cannula 34. Specifically, the alignment ridge 35 is aligned with the openings 37. Thus, a surgeon can ascertain the orientation of the openings 37 when the distal end 39 of the delivery device 10 is in place within the patient by looking at or feeling the alignment ridge 35 of the connection and alignment piece 32. Alternative mechanisms for providing an alignment indication may alternatively be used.

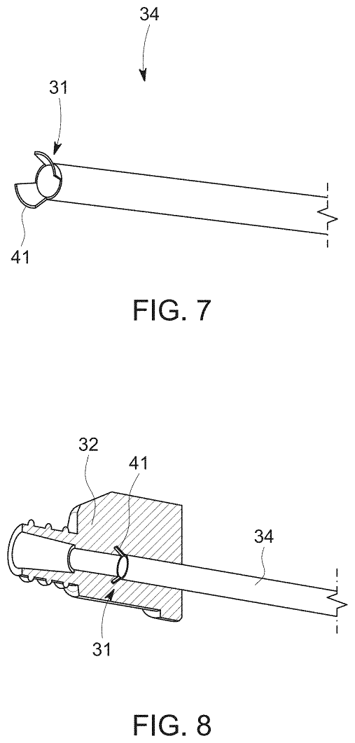

The proximal end 31 of the cannula may be fixed to the inside of the connection and alignment piece 32. This may be done by overmolding the connection and alignment piece 32 onto the proximal end 31 of the cannula. FIG. 7 illustrates the proximal end 31 of the cannula 34 of a cannula assembly of a delivery device, in accordance with one embodiment. As shown, the proximal end 31 may be swaged with flanges 41. FIG. 8 illustrates a connection and alignment piece 32 fit to the proximal end 31 of the cannula 34 of the cannula assembly of a delivery device, in accordance with one embodiment. As shown, the connection and alignment piece may be overmolded onto the swaged end 31.

FIG. 9 illustrates a hub 24 of a push rod assembly 20 of a delivery device and a connection and alignment piece 32 of a cannula assembly 30 of a delivery device, before attachment, in accordance with one embodiment. As shown, the delivery device may include a latching mechanism that allows the push rod assembly 20 and the cannula assembly 30 to be locked together and for easy disengagement of the push rod assembly 20 and the cannula assembly 30. The latching mechanism may be a low profile latching mechanism. In one embodiment, the connection and alignment piece 32 may have a toggle 40 for releasable engagement with a catch 42 on the hub 24. As shown, the catch 24 may be a ramped catch. The push rod 22 of the push rod assembly 20 may be extended through the connection and alignment piece of the cannula assembly 30.

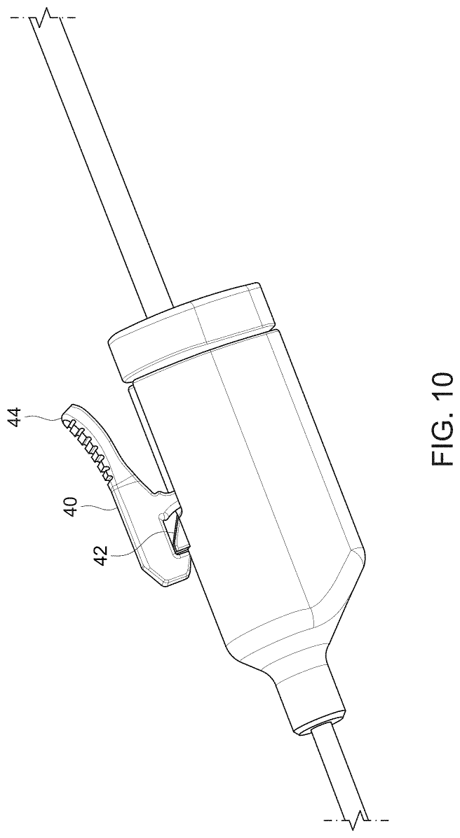

FIG. 10 illustrates a closer view of the hub 24 of the push rod assembly and the connection and alignment piece 32 of the cannula assembly 22 as attached to form the delivery device, in accordance with one embodiment. As shown, the toggle 40 is releasably engaged with the catch 24. The toggle 40 includes a thumb piece 44. Pressing on the thumb piece 44 lifts the toggle 40 and releases it from the catch 24.

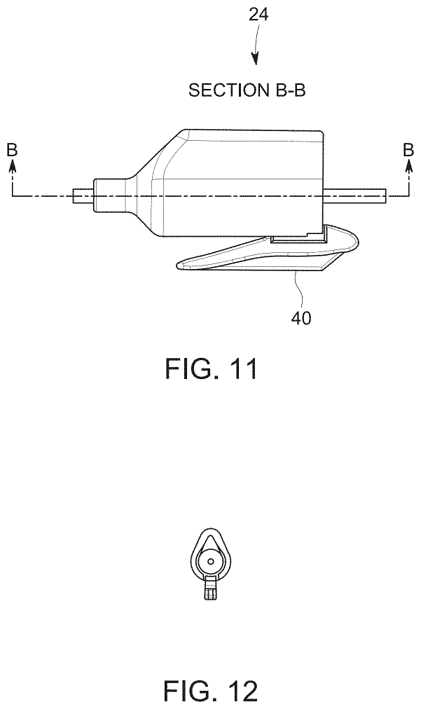

FIG. 11 illustrates an alternative embodiment of the hub 24 and toggle 40 of the delivery device.

FIG. 12 illustrates an end view of the hub assembly.

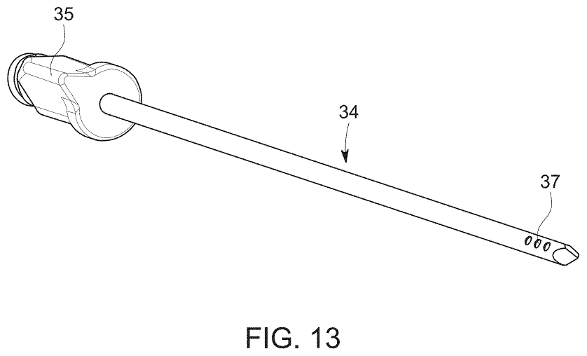

FIG. 13 illustrates an assembled delivery device 10. As shown, the alignment ridge 35 aligns with openings 37 at the distal end 39 of the cannula 34.

In various embodiments, each of the push rod assembly and the cannula assembly comprise a plastic piece over-molded onto a stainless steel piece. In alternative embodiments, the push rod assembly and cannula assembly may be otherwise formed.

Using the Delivery Device

Surgeons can use the delivery device to deliver a flowable material to subchondral lesions in human bone. In summary, a distal end of the delivery device is inserted into subchondral bone. This may be done using a powered wire driver, for example. The push rod assembly is removed, allowing the surgeon to fix a syringe of flowable material onto the cannula portion of the device. The flowable material is then injected into the subchondral lesion. The procedure augments areas of soft bone to reestablish a foundation for the hard bone and cartilage that sit on the bone. This staves off the need for larger surgeries. A more detailed description is provided below.

Use of the delivery device will now be described in more detail. In treatment of subchondral defects, a surgeon assembles the delivery device by inserting the connection and alignment piece 32 of the cannula assembly 20 into the receptacle 19 of the hub 24 of the push rod assembly 10. In one embodiment, the cannula assembly 20 and push rod assembly 10 are latched together by engaging the toggle 40 of the connection and alignment piece 32 with the catch 42 on the hub 24. Other mechanisms for latching the cannula assembly and push rod assembly together may alternatively be used.

A wire piece 22 extending proximally from the hub 24 may be put on a wire driver to drive the cannula into the bone. Under fluoroscopic imaging, the physician targets towards the area to be treated with biologic material, such as bone cement. The cannula is pushed through hard bone into soft, cancellous bone, where lesions are present, under power.

Once the tip is in place, the toggle 40 is released from the catch 42 to disengage the push rod assembly 10 from the cannula assembly 20 and remove the push rod assembly 10. This leaves the cannula 34 in the bone. A syringe is engaged with the connector of the cannula assembly 20. This may be done, for example, via threading of a luer lock. With the syringe engaged, the physician pushes biologic material, for example up to about 3 ccs of bone cement or calcium phosphate, into the cannula, which then exits the device. This can be done through openings 37 and/or through open distal end of the cannula. The physician can adjust the delivery device to align the openings 37 with the area of bone to be treated by using the alignment ridge 35 of cannula assembly 20. The alignment ridge gives the physician a tactile and visual indication of position of the openings 37.

The push rod assembly 10 may be reengaged with the cannula assembly 20 and the push rod pushed to expel any excess material into the bone. The delivery device may then be kept in place for approximately 5 minutes to allow the material to set. After the material is set, the driver can be put in reverse and the delivery device pulled out.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A delivery device for delivering a biologic material, the delivery device comprising:
a push rod assembly;
a cannula assembly comprising a cannula, wherein biologic material may be directed through the cannula and out of an opening provided at or near a distal end of the cannula; and
a connection and alignment piece for detachably connecting the push rod assembly and the cannula assembly, wherein the connection and alignment piece includes an alignment mechanism that is configured to tactilely index a location of the opening and a connector configured for engagement with a syringe.

2. The delivery device of claim 1, wherein the push rod assembly comprises a push rod and a hub.

3. The delivery device of claim 2, wherein the connection and alignment piece is part of the cannula assembly and at least part of the connection and alignment piece is received by a receptacle of the hub of the push rod assembly.

4. The delivery device of claim 3, wherein the connection and alignment piece and the receptacle have complementary cross sections.

5. The delivery device of claim 2, wherein the push rod comprises a heavy gauge wire and a fixation element, the fixation element being braised onto the push rod and molded into the hub.

6. The delivery device of claim 2, wherein the push rod extends proximally and distally from the hub.

7. The delivery device of claim 2, wherein the hub comprises grip features and a catch.

8. The delivery device of claim 1, wherein the alignment mechanism is an alignment ridge that indexes the opening.

9. The delivery device of claim 1, wherein the cannula is a fenestrated cannula wherein the opening is along a surface of the cannula.

10. The delivery device of claim 1, wherein the opening is at an end of the cannula such that the cannula is open.

11. The delivery device of claim 1, further comprising a wire piece extending proximally from the hub.

12. The delivery device of claim 1, further comprising a radiopaque marker at or near a distal end of the cannula.

13. The delivery device of claim 1, wherein the connection and alignment piece is overmolded onto a proximal end of the cannula.

15. The delivery device of claim 1, wherein the cannula assembly is detachably connected to the push rod assembly using a latching mechanism.

16. A delivery device for delivering a biologic material, the delivery device comprising:
a push rod assembly comprises a push rod and a hub;
a cannula assembly comprising:
a cannula, wherein biologic material may be directed through the cannula and out of an opening provided on a surface of the cannula at or near a distal end of the cannula, wherein the cannula is closed and has a trocar tip;
a connection and alignment piece, wherein the connection and alignment piece includes an alignment ridge that is configured to tactilely index a location of the opening and a connector configured for engagement with a syringe; and
a latching mechanism for releasably engaging the push rod assembly.

17. A method of delivering a flowable material, the method comprising:
providing a delivery device, the delivery device comprising:
push rod assembly;
a cannula assembly comprising a cannula, wherein biologic material may be directed through the cannula and out of an opening provided at or near a distal end of the cannula, wherein the cannula assembly may be detachably connected to the push rod assembly; and
a connection and alignment piece for joining the push rod assembly and the cannula assembly, wherein the connection and alignment piece includes an alignment mechanism that is configured to tactilely index a location of the opening and a connector configured for engagement with a syringe;
joining the push rod assembly and the cannula assembly;
driving the cannula into bone;
releasing the push rod assembly from the cannula assembly;
engaging the syringe with the connector of the cannula assembly;
using the alignment piece to align the opening with an area of bone to be treated; and
expelling material from the syringe to the area of bone.

18. The method of claim 17, wherein the cannula is a fenestrated cannula wherein the opening is along a surface of the cannula.

19. The method of claim 18, wherein the cannula includes a trocar tip and wherein driving the cannula into bone is done using the trocar tip.

20. The method of claim 17, wherein the opening is at an end of the cannula such that the cannula is open.
